(19) United States
(12) Patent Application Publication
(10) Pub. No.: US 2024/0041941 A1
(43) Pub. Date: Feb. 8, 2024

(54) METHOD OF TREATMENT FOR HEART FAILURE USING STEM CELLS

(71) Applicant: University of Louisville Research Foundation, Inc., Louisville, KY (US)

(72) Inventor: Roberto Bolli, Louisville, KY (US)

(21) Appl. No.: 18/230,278

(22) Filed: Aug. 4, 2023

Related U.S. Application Data
(60) Provisional application No. 63/395,100, filed on Aug. 4, 2022.

(51) Int. Cl. A61K 35/51; A61P 9/10

(52) U.S. Cl. CPC A61K 35/51; A61P 9/10

(57) ABSTRACT

Methods to treat heart failure and reduce systemic inflammation are provided. The methods include systemically delivering to a subject at least two doses, for example 2-10 doses, of mesenchymal stem cells, wherein the at least two doses are separated by an interval of time, such as from 1 day to 1 year.

### CROSS-REFERENCE TO RELATED APPLICATIONS

This application claims the benefit of U.S. application 63/395,100, filed Aug. 4, 2022. This application is incorporated herein by reference in its entirety.

### STATEMENT REGARDING FEDERALLY SPONSORED RESEARCH OR DEVELOPMENT

This invention was made with government support under grant number P01 HL-78825 awarded by the National Institutes of Health. The government has certain rights in the invention.

### FIELD OF THE INVENTION

The invention relates to a cell therapy method to treat heart failure and reduce systemic inflammation.

### BACKGROUND OF THE INVENTION

Currently, bone marrow derived stem cells have been given in an attempt to heal heart tissues through an immunomodulatory mechanism and treat ischemic heart failure. Historic approaches have relied on single local delivery to the myocardium via intracoronary, transendocardial, or even transepicardial approaches. There is a great need to improve outcomes for patients and devise therapies in this space.

### SUMMARY

The present disclosure provides novel methods for treating heart failure and reducing systemic inflammation by administering multiple doses of mesenchymal stem cells via systemic delivery.

One aspect of the disclosure provides a method for treating heart failure in a subject in need thereof, comprising systemically delivering to the subject at least two doses of mesenchymal stem cells, wherein said at least two doses are separated by an interval of time. In some embodiments, the mesenchymal stem cells are derived from any human tissue. In some embodiments, the human tissue is an umbilical cord. In some embodiments, the mesenchymal stem cells are derived from umbilical cord blood or bone marrow. In some embodiments, the heart failure is either non-ischemic, ischemic, or chronic heart failure.

In further embodiments, the at least two doses are delivered via intravenous delivery. In some embodiments, the at least two doses is between two and ten doses, for example four doses. In some embodiments, the interval of time is between one day and one year, for example two months. In some embodiments, a cell concentration of each dose is between 0.5 to 5 million cells per kg of weight, for example 1 million cells per kg of weight.

Another aspect of the disclosure provides a method of reducing systemic inflammation in a subject in need thereof, comprising systemically delivering to the subject at least two doses of mesenchymal stem cells, wherein said at least two doses are separated by an interval of time.

### BRIEF DESCRIPTION OF THE DRAWINGS

FIG. 1. Effect of multiple vs. single doses on LV function after MI.

FIG. 2. Growth of UC-MSCs vs. BM-MSCs in culture. With these doubling times, after 1 week of culture 1 million BM-MSCs will yield 7.9 million cells, whereas 1 million UC-MSCs will yield >4 times more (30.7 million cells).

FIG. 3. Schematic flowchart of the CATO trial.

### DETAILED DESCRIPTION

Embodiments of the disclosure provide for the treatment of heart failure, for example non-ischemic, ischemic, or chronic heart failure and the reduction of systemic inflammation via systemic delivery (e.g. intravenous delivery) of mesenchymal stem cells. The rationale for this mode of delivery is both pathophysiological and practical.

Pathophysiological rationale. For the past 20 years, studies of cell therapy have been based on the assumption that transplanted cells work by i) differentiating into new myocytes, ii) promoting new myocyte formation from endogenous sources, and/or iii) releasing paracrine factors that favorably affect the surrounding host tissue. Accordingly, every effort has been made to deliver cells directly to the heart. Since i.v. injection results in negligible cardiac retention of cells (vide infra),¹¹⁻¹⁵ local delivery to the myocardium via intracoronary, transendocardial, or even transepicardial approaches has been used in almost every preclinical and clinical study to date.¹⁶

However, in recent years the traditional assumption that only cells delivered directly to the heart are effective has been challenged by two lines of evidence. First, i.v. administration of MSCs has been repeatedly shown to impart beneficial effects in numerous animal studies^{12-15, 17-26, 27-31} and in two Phase I clinical trials of HF^{32, 33} (vide infra). Second, mounting evidence indicates that the progressive deterioration in left ventricular (LV) function after myocardial infarction (MI) (ischemic cardiomyopathy [ICM]) is caused, at least in part, by persistent systemic inflammation resulting from activation of the immune system and infiltration of the myocardium by inflammatory cells (e.g., monocytes).³⁴⁻³⁷ The spleen in particular has been implicated as an important component of this response (cardiosplenic axis).³⁴⁻³⁷ A corollary of this emerging concept is that interventions that suppress systemic inflammation may alleviate myocardial inflammation and post-MI LV dysfunction.¹⁶ MSCs (whether derived from bone marrow (BM), umbilical cords (UC), or other tissues) possess immunomodulatory and anti-inflammatory actions, including suppression of native and adaptive immunity.^{16, 38-41} These actions have been demonstrated not only in animal and human studies of ischemic HF (e.g., in patients with HF, MSCs reduce circulating TNF-α levels 5) but also in many clinical noncardiac conditions in which MSCs have been found to modulate immune responses in patients with various disorders.^{41, 42} Systemic delivery of MSCs targets the spleen and other immune tissues more effectively than local cardiac delivery, and indeed, MSCs trapped in extracardiac tissues (lungs, spleen) have been shown to exert systemic anti-inflammatory actions.^{16, 43} Embodiments of the present disclosure are targeted at reducing systemic inflammation with systemic (i.v.) administration of MSCs to alleviate ischemic HF.

Intravenous delivery of cells offers significant advantages over local cardiac delivery, which is by necessity invasive. Thus far, most trials of cell therapy in HF have used transendocardial injections to deliver cells.¹⁰ Although this delivery route is effective (as attested by many “positive” studies^{3, 10, 16}), it has important limitations with respect to widespread adoption of cell therapy, particularly for repeated treatments. Transendocardial injections are expensive, labor intensive, require specialized training and facilities, carry significant risks (e.g., ventricular tachyarrhythmias, left bundle branch block, LV wall perforation, hemopericardium, death), and cannot be performed in patients with LV assist devices (LVADs), prosthetic valves, LV cavity thrombi, induced tachyarrhythmias, permanent atrial fibrillation with uncontrolled rate, or frequent irregular ectopy. Since cells can be delivered only to a limited number of LV sites (rather than to the entire left ventricle), the paracrine actions of the cells are probably restricted to the injected sites. Furthermore, as detailed below, there is mounting evidence that transplanted cells are rapidly cleared from the heart.^{16, 45-50} This problem of rapid cell disappearance is universal: it has been observed with virtually all cell types⁴⁵ and thus is a major factor that limits the efficacy of all forms of cell therapy tested heretofore.^{16, 45, 49} However, the transendocardial route does not lend itself to repeated administrations because repeated injections would carry significant risks and reduce patient acceptance, and thus would not be feasible (this is a major reason why virtually all clinical studies of cell therapy in HF performed heretofore have used one cell administration^{10, 16, 45, 49}). Also, if a clinical trial of multiple cell administrations were conducted, a placebo group undergoing repeated transendocardial injections would not be ethically justifiable, making blinding impossible.

In contrast, i.v. infusions are simple, inexpensive, carry significantly less risk, and can be performed almost everywhere in almost all patients with HF as outpatient procedures. For these reasons, patient acceptance and adherence are higher. The paracrine or endocrine actions of systemically delivered cells affect the entire left ventricle rather than selected myocardial foci, and thus may be quite effective. Finally, the i.v. route is exquisitely suitable for repeated infusions of cells, even for numerous doses. Thus, the i.v. route lends itself not only to widespread clinical utilization, but also to repeated cell administrations.

Hence, there is long felt unmet need in the market for a more practical solution to delivery of cell population that is both easier to administer and more effectively treats inflammatory complications of HF. Our innovative method of multiple doses of a particular population of MSCs addresses this gap.

The mesenchymal stem cells described herein may be derived from any human tissue. In some embodiments, the human tissue is an umbilical cord. In some embodiments, the mesenchymal stem cells are derived from umbilical cord blood or bone marrow.

Numerous preclinical studies in small^{12-15, 17-26} and large (porcine)²⁷⁻³¹ animal models of acute MI or chronic ICM (old MI) (19 investigations, including 5 pig studies) have investigated i.v. delivery of MSCs. All of these studies have shown improvement in LV function despite the fact that MSCs do not engraft in the heart.¹⁶ Thus, there is considerable preclinical evidence that i.v. administration of MSCs alleviates post-MI LV dysfunction. The mechanism of action may involve systemic anti-inflammatory and immunomodulatory actions of MSCs leading to reduced myocardial inflammation.^{16, 39} After i.v. infusion, significant MSC entrapment has been found in the lungs and, to a lesser extent, in other organs such as the spleen;^{14, 15, 27, 28, 31, 51, 52} in contrast, few MSCs (<3%) homed to the heart, where they disappeared within few days.^{14, 15, 28, 31, 51, 52} This implies that the beneficial effects on LV function were not due to engraftment of cells in the heart but instead to systemic actions of cells that lodged in extracardiac organs (e.g., spleen).¹⁶

MSCs exert immunomodulatory effects in vitro.^{16, 38-41} In addition, several in vivo studies have documented that i.v. infusion of MSCs (derived from BM, adipose tissue, or UC) results in decreased myocardial infiltration by macrophages, decreased myocardial expression of pro-inflammatory cytokines (e.g., IL-1β and IL-6), and increased expression of anti-inflammatory cytokines (e.g., IL-10).^{14, 24, 25} Transplanted cells trapped in the lungs (and other organs) have been shown to release immunomodulatory signals that produce systemic anti-inflammatory effects leading to reduced myocardial inflammation.^{14, 53, 54} Since some of the i.v. injected MSCs home to the spleen^{14, 23} and various lymphoid and non-lymphoid organs,^{14, 15, 27, 28, 31, 51, 52} it is conceivable that they can also modulate the locally residing host immune cells, which in turn may affect systemic and local myocardial inflammation.¹⁶

Taken together, the available evidence suggests that after i.v. infusion, MSCs lodged in the lung, spleen, and other extracardiac tissues produce systemic anti-inflammatory effects either via paracrine actions on adjacent immune cells which are then released into the circulation or via endocrine release of anti-inflammatory factors into the blood. In addition to these anti-inflammatory actions, MSCs secrete a panoply of extracellular vesicles, cytokines, non-coding RNAs, etc.;⁴¹ release of these factors from extracardiac sites into the circulation may exert other actions that are beneficial to the infarcted heart, e.g., anti-apoptotic, angiogenic, and anti-fibrotic actions.¹⁶ Regardless of the precise mechanism of action, the consistent efficacy observed in preclinical studies provides a strong rationale for the usefulness of the present disclosure.

Many clinical trials have shown that i.v. infusion of allogeneic MSCs is safe and does not elicit immune rejection against the transplanted cells,^{4, 5, 32, 33, 55-69} even when repeated multiple times.^{55, 57, 59-61, 66-68} This has been reported after administration of MSCs (i.v. or transendocardially) not only in the cardiovascular arena^{4, 5, 32, 33, 65, 69} but also in noncardiovascular conditions.^{55-64, 66-68} Safety and lack of immune reactions to injected MSCs have been confirmed by meta-analyses.⁷⁰⁻⁷¹ The concept that i.v. MSCs exert systemic anti-inflammatory actions³³ is consistent with the well-known immunomodulatory properties of these cells^{16, 38-41} and has been clearly demonstrated by many clinical trials showing systemic anti-inflammatory and immunomodulatory actions of i.v. MSC therapy in various conditions, including HF,⁵ graft-versus-host disease, multiple sclerosis, amyotrophic lateral sclerosis, systemic lupus erythematosus, chronic obstructive pulmonary disease, and Crohn's disease.⁵⁵⁻⁶⁰

Three randomized Phase I clinical trials of i.v. cell therapy have been reported in patients with heart disease. Hare et al.⁶⁵ allocated 53 patients with acute MI (STEMI) to i.v. allogeneic BM-MSCs or placebo. No difference in adverse event rates was observed between the two groups. In the subset of patients with an anterior MI, LVEF improved at 6 months after treatment with BM-MSCs but not in controls. This double-blinded trial provided evidence of safety, and provisional evidence of efficacy, of i.v. MSCs in patients with acute MI.

The RIMECARD trial by Bartolucci et al.³² was a Phase I, randomized, double-blind, and placebo-controlled study in which patients with ischemic or nonischemic cardiomyopathy received an i.v. infusion of UC-MSCs (1×106 cells/kg) or placebo (n=15 per group) and were followed for 1 year. No adverse events were reported, suggesting again that i.v. delivery of cells was safe. None of the patients tested developed alloantibodies to the injected cells. Compared with baseline values, patients given UC-MSCs, but not those receiving placebo, exhibited an improvement in LVEF (assessed both by echocardiography and MRI) vs. baseline that became apparent at 3 months and was sustained at 6 and 12 months. Throughout the follow-up period, the NYHA class and KCCQ score all improved in the UC-MSC-treated group, but not in the control group. In vitro studies demonstrated that UC-MSCs and BM-MSCs produced similar degrees of inhibition of T cell proliferation, suggesting similar immunosuppressive properties. However, although the secretomes of the two cell types were similar, UC-MSCs exhibited much higher expression of hepatocyte growth factor (HGF) and greater migration capacity.

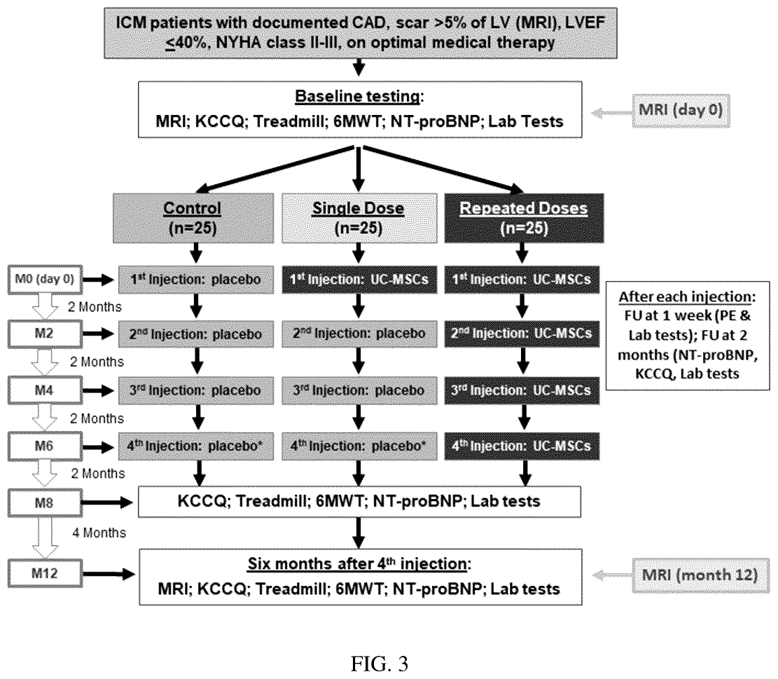

In summary, all of these trials^{32, 33, 65} have provided encouraging results with respect to efficacy (improved LV function and functional capacity) and safety after i.v. infusion of allogeneic MSCs and have shown that no humoral immune reaction develops. However, no trial of i.v. cell therapy has been conducted heretofore specifically in patients with ischemic HF and no trial has studied multiple infusions of cells with extended times between doses. Furthermore, no study has investigated the mechanism of action of i.v. cell therapy in cardiac patients. Described herein is a proposed larger, rigorous Phase II study (named “CATO”) which will explore the mechanism of action (FIG. 3).

The use of the i.v. route (a paradigm shift) will enable us to administer multiple cell doses (another paradigm shift). Indeed, as mentioned above, it would be very difficult or impossible to give multiple doses using intracoronary or transendocardial routes. One novel aspect of this invention is the multiple dosing of cells via i.v. administration of cells. The dosing schedule of cells will be separated by at least 7 days and up to one year, for example at intervals of 1 to 12 months, e.g. every 2 to 4 months. It is contemplated that 3-10 such doses, e.g. about 4 doses, will effectively treat ischemic heart failure. And for chronic disease management, one or several ongoing treatments/year may be optimal to treat chronic disease.

A patient or subject to be treated by any of the compositions or methods of the present disclosure can mean either a human or a non-human animal including, but not limited to dogs, horses, cats, rabbits, gerbils, hamsters, rodents, birds, aquatic mammals, cattle, pigs, camelids, and other zoological animals.

In some embodiments, the composition is administered to the subject in a therapeutically effective amount. By a “therapeutically effective amount” is meant a sufficient amount to treat the disease or disorder at a reasonable benefit/risk ratio applicable to any medical treatment. It will be understood that the total daily usage of the compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific active agent employed; and like factors well known in the medical arts. For example, it is well within the skill of the art to start doses of the compound at levels or frequencies lower than those required to achieve the desired therapeutic effect and to gradually increase the dosage or frequency until the desired effect is achieved. The optimal cell dose will be in the range of 0.5-5 million cells/kg with a preferred number of 1 million/kg.

In the most preferred embodiment, UC MSC's will be delivered to patients at a rate of 0.5 to 5 million cells per kilogram of patient weight in at least four doses separated by at least 2 months. The time to separate the doses shall be determined empirically and multiple dosing to control the immune dysfunction of the patient is one key aspect of the invention. Functional assays on the patient heart health may be used to guide the timing of subsequent doses of MSC's.

Almost all preclinical and clinical studies of cell therapy performed in the past two decades have used one administration of a cell suspension. However, as mentioned above, in recent years it has become clear that cells disappear rapidly (within few weeks) after transplantation.^{16, 49} For example, after intracoronary or intramyocardial injection of c-kit-positive cardiac cells (CPCs) in mice, <3% of the CPCs remain in the heart 35 days later.⁵⁰ This rapid disappearance is a universal problem that has been observed with virtually all cell types⁴⁵ and thus is a major factor that limits the efficacy of all forms of cell therapy tested heretofore. Particularly in a chronic condition such as HF, which develops over years, it is difficult to envision how a single treatment could have sustained effects.

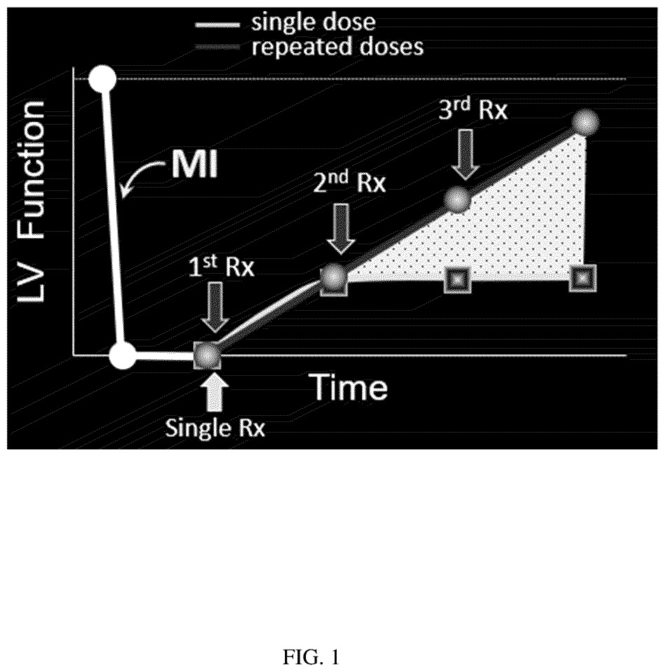

It is contemplated herein that the use of a single dose is a major reason for the borderline or negative results of previous clinical trials^{10, 45, 49} and that for the full therapeutic effects to become apparent, it is necessary to use repeated doses in order to replace the cells that disappear and to sustain an immunologic effect. Thus, in CATO we will demonstrate the concept that the rapid disappearance of cells can be overcome by administering repeated cell doses (FIG. 3). Our fundamental hypothesis (illustrated in FIG. 1) is that the beneficial effects of cell therapy are significantly augmented by repeated cell administrations.

The protocol for CATO is illustrated in FIG. 3. Briefly, 60 subjects will be assigned in a random fashion to three groups on a 1:1:1 basis:control (placebo), single dose, and repeated doses. After randomization and baseline testing, subjects will receive four SPIs via the i.v. route at 2-month intervals. SPIs will consist of either 100×10⁶ UC-MSCs or placebo (Plasma-Lyte A with 1% HSA). Subjects in the control group will receive 4 doses of placebo. Subjects in the single-dose group will receive 1 dose of UC-MSCs followed by 3 doses of placebo. Subjects in the repeated-dose group will receive 4 doses of UC-MSCs. Placebo will be infused i.v. at a rate of 1 ml/min for a total of 60 ml over 60 min. UC-MSCs (100×10⁶ cells) will be infused i.v. at a rate of 1 ml/min for a total of 60 ml over 60 min (1.67 million cells/ml/min). After each SPI, subjects will be monitored for 4-6 hours, examined at 1-week and 2-month visits, and contacted at 1 month for a telephone assessment. All subjects will be examined at six months after the last (fourth) SPI to complete safety and efficacy assessments.

To assess LV function, structure, and scar size, two MRI scans will be performed: at baseline (M0) and 6 months after the fourth SPI (M12). NYHA class, KCCQ score, and laboratory testing will be assessed at baseline (M0), 2 months after each SPI (M2, M4, M6, M8), and at the end of follow-up (M12). Functional capacity (VO₂ max and 6MWT) will be assessed at baseline (M0) and 6 months after the fourth SPI (M12).

Preclinical studies of repeated cell doses. It is contemplated that the beneficial effects of repeated cell doses cannot not be explained by engraftment and differentiation of transplanted cells, and thus must reflect paracrine mechanisms, possibly anti-inflammatory actions.¹⁶ Importantly, the cumulative effects of three repeated doses could not be recapitulated by a single combined dose, even though the total number of cells infused was the same, suggesting that the duration of myocardial exposure to the transplanted cells is more important than the intensity of such exposure^{48, 77}.

Clinical studies of repeated cell doses. There is limited clinical experience with the use of repeated cell doses in patients with heart disease. A randomized trial of 39 patients with STEMI given intracoronary BM-derived mononuclear cells (BM-MNCs) at 3-7 days after MI found that a second intracoronary infusion of BM-MNCs 3 months later resulted in decreased scar size and improved LVEF.⁷⁸ An open-label, nonrandomized study of 32 patients with ischemic HF reported improved LV filling (assessed by echocardiography) and decreased plasma NT-proBNP levels in patients given 2 doses of BM-MNCs via the intracoronary route 4 months apart.⁷⁹ An open-label, nonrandomized study of patients with recent (<3 months) STEMI who were given G-CSF and 1 or 2 intracoronary infusions of BM-MNCs (6 months apart) (n=15/group) found improved myocardial function and perfusion in the cohort that received 2 infusions.⁸⁰ A registry study of 297 patients with ischemic HF suggested that a second intracoronary infusion of BM-MNCs (performed in 111 patients at 3-6 months after the first infusion) results in improved survival.⁸¹ In patients with nonischemic cardiomyopathy, Vrtovec et al.⁸² reported no improvement in LVEF, NT-proBNP levels, or 6-min walking distance after 2 transendocardial injections of CD34+ cells 6 months apart (n=30) vs. a single injection (n=30);⁷⁸ however, although that study was randomized, it was not blinded and lacked a placebo group. All of these studies,⁷⁹⁻⁸² as well as a very large number of studies in noncardiovascular conditions,^{55, 57, 59-61, 66-68} have documented the safety of repeated doses of allogeneic cells.

Although the studies reviewed above⁷⁹⁻⁸² provided initial proof-of-principle, they were limited by the lack of a true control group because, except for the STEMI study,⁷⁸ in all of the other trials the single-dose group did not receive a second (placebo) infusion. None of the aforementioned trials used MSCs, none tested more than two doses of cells, and none used a double-blinded, placebo-controlled design. It is contemplated herein that reducing systemic inflammation with systemic administration of UC-MSCs will alleviate ischemic HF.

Preclinical studies of UC-MSCs. UC-MSCs share the beneficial properties of BM-MSCs, including immunomodulatory, anti-inflammatory, anti-fibrotic, and angiogenic actions as well as low immunogenic potential.^{41, 42, 83-88} Compared with BM-MSCs, however, UC-MSCs express lower levels of HLA-class I, appear to have greater immunosuppressive effects, exhibit less cellular aging, have faster growth rates, do not require invasive harvesting procedures, and because of the abundance of donors and higher proliferative rate, are less expensive to produce.^{16, 32, 87, 88} In vitro studies have shown that, compared with BM-MSCs, UC-MSCs exhibit superior clonogenicity, migration, and paracrine actions (e.g., they secrete much larger quantities of cytokines such as HGF)³² Importantly, there is evidence that UC-MSCs are more potent than BM-MSCs (since they are derived from a much younger organism).^{16, 32, 87, 88}

Administration of UC-MSCs has consistently improved LV function and reduced apoptosis and fibrosis in several rodent and pig models of acute MI or chronic ICM.^{16, 87, 89, 84, 85, 90-96} Of note, in a pig model of chronic ICM (similar to the clinical setting in CATO, FIG. 3), administration of UC-MSCs improved LVEF and fractional shortening, increased myocardial perfusion, angiogenesis, and collateral development, and reduced apoptosis and fibrosis.⁹¹ Anti-apoptotic and anti-fibrotic actions of UC-MSCs have also been documented in rodent models of chronic HF.⁸³⁻⁸⁶ UC-MSCs enhance angiogenesis in vitro and in vivo through upregulation of various proangiogenic factors and chemokines, including VEGF, angiopoietin, and MCP-1 among others.⁸³⁻⁸⁶

In summary, UC-MSCs offer numerous potential advantages over BM-MSCs, providing a solid rationale for clinical investigations of UC-MSCs.

Clinical studies of UC-MSCs. As discussed above the Phase I trial by Bartolucci et al.³² (RIMECARD) found i.v. infusion of UC-MSCs to be safe and effective in 30 patients with HF.³² The CATO study described here will use the same UC-MSCs as in the RIMECARD study. That study, however, did not focus specifically on patients with ischemic HF and did not investigate the mechanism of action of UC-MSCs. Several studies of intracoronary infusion of UC-MSCs have been published⁹⁷⁻⁹⁹ A randomized Phase I trial⁹⁷ reported improvement in LVEF and 6-minute walk distance at 6 months after intracoronary infusion of UC-MSCs in patients with chronic HF. In a randomized, double-blind study of 116 patients with STEMI, intracoronary infusion of UC-MSCs increased myocardial viability and LVEF 18 months later.¹⁰⁰ In addition, observational studies in patients with ischemic HF suggest improved LVEF after intracoronary infusion of UC-MSCs.⁹⁸ Although these studies^{97 100 98} support the utility of UC-MSCs, they did not examine the i.v. route of administration. Many other studies have reported beneficial effects and anti-inflammatory actions of UC-MSCs (mostly given by the i.v. route) in various noncardiovascular settings (e.g., multiple sclerosis, spinal cord injury, systemic lupus erythematosus, rheumatoid arthritis, ulcerative colitis, Crohn's disease, and graft-vs.-host disease [GVHD]).^{57, 101-111} For example, a recent study showed that two i.v. infusions of UC-MSCs (108 cells each) improved survival and reduced inflammatory cytokines (GM-CSF, INF-gamma, 11-2, IL-6, TNF-alpha, TNF-beta) in COVID-19 patients (NCT04355728. refs.112, 113). It is because of their profound anti-inflammatory properties that multiple doses of UC-MSCs (2, 3, or 4 doses) are being given i.v. (108 cells or similar number/dose) in a multitude of ongoing studies to patients with COVID-19 (at least 30 studies, e.g., NCT04269525, NCT04273646, NCT04313322, NCT04273646, NCT04399889, NCT04494386, NCT04355728, NCT04400032, NCT04333368, NCT05286255), cerebral palsy (NCT03473301), T2D (IND #018302), or Alzheimer disease (IND #18200) (reviewed in ref. 112). All of the above studies have consistently found that UC-MSCs are safe and do not elicit a humoral immune reaction.^{32, 57, 97, 100-112, 114}

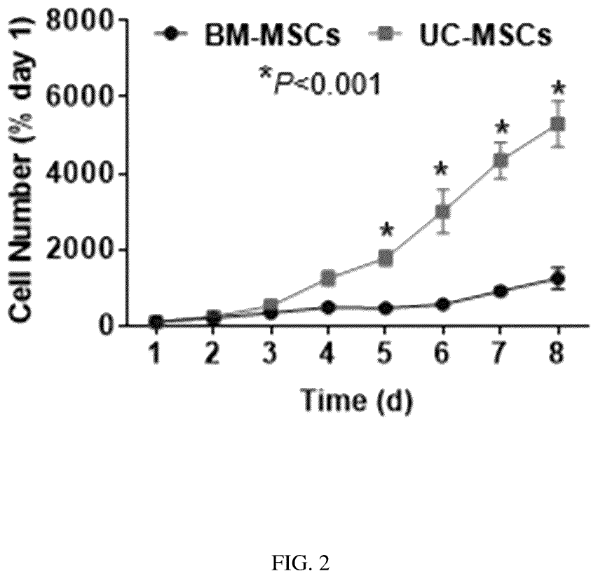

Characterization of the cell product in CATO. UC-MSCs exhibit a much shorter doubling time (average, 33.9 h vs. 56.5 h in BM-MSCs, −40%, P<0.01) and much higher positivity for markers of proliferation (EdU incorporation, Ki67, P-histone H3; P<0.01), indicating a much more robust proliferative activity. With these doubling times, after 1 week of culture 1 million BM-MSCs will yield 7.9 million cells, whereas 1 million UC-MSCs will yield >4 times more (30.7 million cells) (FIG. 2). If a similar difference exists in vivo after transplantation, the number of cells available to repair the heart would be much greater with UC-MSCs than BM-MSCs. Furthermore, the greater proliferative capacity of UC-MSCs means that fewer passages are needed to achieve the target cell number and younger cells will be injected into patients. Consistent with the above, nuclear size and markers of senescence (beta-Gal activity, p21 positivity) were significantly less in UC-MSCs after 4-6 passages, indicating that the development of senescence is delayed in UC-MSCs. Furthermore, analysis of 116 cytokines in conditioned media showed much more abundant secretion by UC-MSCs of cytokines known to modulate immune responses, angiogenesis, and LV remodeling in HF, e.g., follistatin, 116 GCP-2,117 G-CSF, 118 GRO-alpha, 119 IL-6,120 IL-8,121 MCP-1,122 PDGF-AA, 123 and TGF-beta 124 (Table 1). The superior immunomodulatory properties of UC-MSCs vs. BM-MSCs were also confirmed by extensive RNA Seq analyses of the two cell types. These observations, together with the consistent anti-inflammatory actions of UC-MSCs repeatedly observed in vivo both in animal models and in humans clearly demonstrate the anti-inflammatory properties of these cells. Together, these data show that the cell product to be used meets the criteria for mesenchymal cells and suggest that it is potentially superior to BM-MSCs.

TABLE 1
Comparison of UC-MSCs and BM-MSCs
Immunophenotype

| | UC-MSCs | BM-MSCs |
|---|---|---|
| | (n = 3) | (n = 3) |
| CD73 | 99.50 ± 0.50 | 100.0 ± 0 |
| CD90 | 99.60 ± 0.40 | 100.0 ± 0 |
| CD105 | 93.73 ± 5.68 | 99.97 ± 0.03 |
| CD45 | 1.77 ± 0.58 | 0.98 ± 0.32 |
| CD19 | 2.64 ± 0.69 | 1.11 ± 0.33 |
| CD11b | 3.49 ± 0.77 | 2.14 ± 0.37 |
| HLA-DR | 0.42 ± 0.10 | 4.16 ± 3.72 |
| CD79a | 0.39 ± 0.13 | 0.36 ± 0.13 |
| CD34 | 1.37 ± 0.20 | 1.46 ± 0.51 |

Proliferative Capacity

| | UC-MSCs | BM-MSCs |
|---|---|---|
| | (n = 6) | (n = 6) |
| Doubling time (h) | 33.93 ± 0.97 | 56.49 ± 3.82** |
| EdU positivity (%) | 41.24 ± 3.01 | 9.99 ± 3.5** |
| Ki67 positivity (%) | 84.86 ± 2.54 | 36.56 ± 8.16** |
| P-histone H3 (%) | 1.56 ± 0.14 | 0.68 ± 0.12** |

Cell Senescence

| | UC-MSCs | BM-MSCs |
|---|---|---|
| | (n = 6) | (n = 6) |
| Nuclear size (μm) | 211.4 ± 11.36 | 248.8 ± 8.39* |
| β-Gal activity (A.U.) | 212.1 ± 27.75 | 473.1 ± 49.08** |
| p21 positivity (%) | 38.97 ± 1.79 | 63.8 ± 3.49** |

Cytokines in conditioned media

| | UC-MSCs | BM-MSCs |
|---|---|---|
| | (n = 3) | (n = 3) |
| Follistatin | 265.1 ± 230.4 | 52.7 ± 42.9 |
| GCP-2 | 1621.5 ± 262.6 | 135.9 ± 131.3** |
| G-CSF | 335.8 ± 425.4 | 24.3 ± 11.9 |
| GRO-alpha | 5584.4 ± 529.3 | 71.0 ± 40.5** |
| IL-6 | 3370.3 ± 1627.2 | 327.7 ± 80.1* |
| IL-8 | 19588.6 ± 10684.5 | 147.8± 56.2* |
| MCP-1 | 170.2 ± 113.2 | 28.1 ± 15.3 |
| PDGF-AA | 135.8 ± 97.7 | 7.1 ± 3.3 |
| TGF-beta2 | 346.4 ± 64.2 | 95.3 ± 47.0** |

*P < 0.05,
**P < 0.01.

As shown in Table 1, we found that both cell types express the classic markers of MSCs and have similar surface antigens. However, UC-MSCs exhibit a much shorter doubling time (average, 33.9 h vs. 56.5 h in BM-MSCs, −40%, P<0.01) and much higher positivity for markers of proliferation (EdU incorporation, Ki67, P-histone H3; P<0.01), indicating a much more robust proliferative activity.

As pointed out below, the range of cell numbers used heretofore for a single i.v. infusion of UC-MSCs is relatively narrow, ranging from 1.0×10⁶ cells/kg³² (i.e., ˜80×106 cells) to 100×10⁶ (NCT03059355) and 150×10⁶ cells.^{57, 101-111} The dose of UC-MSCs to be given i.v. in CATO (100×10⁶ cells) is similar to that given i.v. in the RIMECARD study³² (1.0×10⁶ cells/kg), where UC-MSCs were found to have beneficial effects, and is the same as that used in a recent study of ARDS in COVID-19 patients, where UC-MSCs reduced inflammation and improved survival.¹¹² This dose is also similar to the doses of UC-MSCs used in CERES (NCT03059355), in many of the aforementioned noncardiovascular studies^{57, 101-111} (where i.v. infusion of UC-MSCs was beneficial), and in many ongoing studies of patients with COVID-19 (e.g., NCT04269525, NCT04273646, NCT04313322, NCT04273646, NCT04399889), cerebral palsy (NCT03473301), T2D (IND #018302), or Alzheimer disease (IND #18200) (reviewed in ref.112). Our dose of UC-MSCs is also similar to the dose of BM-MSCs given i.v. by Butler et al.³³ in HF patients (1.5×10⁶ cells/kg).

Recently, Hare et al. administered three doses of BM-MSCs (20, 100, and 200×10⁶ cells) i.v. in patients with aging frailty (CRATUS trial).^{125, 126} They found that physical performance improved significantly in the 100×10⁶ group but not in the 200×10⁶ group, whereas immunologic improvement occurred in both groups, suggesting that greater cell numbers may not necessarily translate into greater benefit.^{125, 126} Higher doses of UC-MSCs would also raise the concern of pulmonary microembolization and would increase the cost of the trial.

Our use of an optimal 2-month interval between doses is supported by clinical trials of MSCs in noncardiovascular conditions, which have shown that the beneficial effects of one dose of i.v. MSCs become manifest at 2 months but not at 1 month after therapy.^{57, 58, 61, 62}

As mentioned above, there is mounting evidence that a persistent systemic inflammatory state contributes to the progressive deterioration in LV function in ICM34-36 and that UC-MSCs exert immunosuppressive actions.^{41, 42, 83-88} For example, in the RIMECARD study, i.v. delivery of UC-MSCs inhibited proinflammatory T cells in vitro;³² in COVID-19 patients, i.v. delivery of UC-MSCs suppressed numerous pro-inflammatory cytokines (GM-CSF, INF-gamma, II-2, IL-6, TNF-alpha, TNF-beta).¹¹² However, the immunologic changes that occur in HF patients in response to cell therapy (either UC-MSCs or any other cell types) have not been systematically analyzed.

The clinical study “CATO” will be a superb opportunity to elucidate the immunomodulatory effects of cell therapy in HF. We will conduct a comprehensive analysis of the effects of UC-MSCs on humoral and cellular immunity, examining not only a panoply of pro- and anti-inflammatory cytokines and chemokines, but also various immune cell types. The insights obtained from this analysis will be highly mechanistic; they will not only elucidate the immunomodulatory actions of UC-MSCs in patients with ischemic HF, but will also illuminate the pathophysiology of ICM. This study will demonstrate the protective immune effects of MSCs that could be beneficial in HF.

Cell Manufacturing.

UC-MSCs will be derived from UC tissue (either gender) obtained from a healthy pregnant woman at the time of caesarean delivery. A single cord will yield approximately 3.5-4 billion cells. The validated process submitted to the FDA describing donor collection processes, infrastructure, and operations under the CMC section of IND #17324. Details regarding testing, screening, and eligibility criteria of donors; collection, expansion, and phenotyping of UC-MSCs; in-process controls; FACS analysis; sterility determination; endotoxin assay; and shipment of cryopreserved UC-MSCs are included in Attachment 7. UC-MSCs will be cryopreserved after 3 passages.

UC-MSC preparation for administration (final formulation). Upon request, the appropriate number of released, frozen bags will be thawed in a 37±1° C. water bath. In a biological safety cabinet, the cell suspension will be transferred to conical tubes and slowly diluted with Plasma-lyte A supplemented with 1% HSA. The diluted suspension will be centrifuged and the cell pellet suspended in the dilution buffer. The cells will be counted to determine total viability. The cells will be centrifuged and the cell pellet resuspended in buffer to a cell concentration of 100 million in 60 ml.

Release criteria will include >80% positivity for CD73, CD90, and CD105 and <2% for lympho-hematopoietic lineage marker CD45+ subsets, including CD34, CD14, CD19, HLA-DR or class II markers (as per ISCT guidelines).¹⁵⁴

Significance. HF is a leading cause of morbidity and mortality and a major public health problem, one that has achieved epidemic proportions. Approximately 6 million Americans suffer from HF and ˜600,000 new cases develop every year.¹ As the population ages, the prevalence of HF continues to increase.¹ Despite optimal medical therapy, the prognosis of HF patients remains poor.¹ Therefore, there is a clear need for new treatment strategies for this deadly syndrome. Although clinical trials of cell therapy for chronic HF have yielded promising results^{6-8, 155-157} (reviewed in ref.^{3, 10, 158}), the development of cell therapy is hindered in part by the invasive nature of current methods for cell delivery (transendocardial or intracoronary injection), which are expensive, complex, carry risks, and do not allow repeated treatments which are necessary in view of the fact that transplanted cells are now known to disappear quickly after delivery regardless of the cell type used.^{16, 49, 45} In addition to this clinical obstacle, there remains a formidable conceptual problem that of chronic treatment for a chronic disease. Current therapy strategies have focused on single interventions.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. A method for treating heart failure in a subject in need thereof, comprising systemically delivering to the subject at least two doses of mesenchymal stem cells, wherein said at least two doses are separated by an interval of time.

2. The method of claim 1, wherein the mesenchymal stem cells are derived from any human tissue.

3. The method of claim 2, wherein said human tissue is an umbilical cord.

4. The method of claim 1, wherein the mesenchymal stem cells are derived from umbilical cord blood.

5. The method of claim 1, wherein the mesenchymal stem cells are derived from bone marrow.

6. The method of claim 1, wherein the heart failure is either non-ischemic, ischemic, or chronic heart failure.

7. The method of claim 1, wherein the at least two doses are delivered via intravenous delivery.

8. The method of claim 1, wherein the at least two doses is between two and ten doses.

9. The method of claim 1, wherein the interval of time is between one day and one year.

10. The method of claim 1, wherein a cell concentration of each dose is between 0.5 to 5 million cells per kg of weight.

11. A method of reducing systemic inflammation in a subject in need thereof, comprising systemically delivering to the subject at least two doses of mesenchymal stem cells, wherein said at least two doses are separated by an interval of time.

12. The method of claim 11, wherein the mesenchymal stem cells are derived from any human tissue.

13. The method of claim 12, wherein said human tissue is an umbilical cord.

14. The method of claim 11, wherein the mesenchymal stem cells are derived from umbilical cord blood.

15. The method of claim 11, wherein the mesenchymal stem cells are derived from bone marrow.

16. The method of claim 11, wherein the at least two doses are delivered via intravenous delivery.

17. The method of claim 11, wherein the at least two doses is between two and ten doses.

18. The method of claim 11, wherein the interval of time is between one day and one year.

19. The method of claim 11, wherein a cell concentration of each dose is between 0.5 to 5 million cells per kg of weight.
